(19) United States
(12) Patent Application Publication
(10) Pub. No.: US 2024/0221863 A1
(43) Pub. Date: Jul. 4, 2024

(54) METHOD AND SYSTEM FOR PREDICTING A BINDING AFFINITY OF PROTEIN STRUCTURES BASED ON DEEP LEARNING

(71) Applicant: Innoplexus AG, Eschborn (DE)

(72) Inventors: Sudhanshu Kumar, Bokaro (IN); Joel Joseph, Kasargod (IN); Ansh Gupta, Sant Kabir Nagar (IN)

(73) Assignee: Innoplexus AG, Eschborn (DE)

(21) Appl. No.: 18/148,474

(22) Filed: Dec. 30, 2022

(51) Int. Cl. G16B 15/30; G06N 3/0464; G16B 15/20; G16B 40/20

(52) U.S. Cl. CPC G16B 15/30; G06N 3/0464; G16B 15/20; G16B 40/20

(57) ABSTRACT

A system and a method for predicting a binding affinity of protein structures based on deep learning is disclosed. The method includes capturing, using a data capture module, a multi-dimensional structure of a plurality of protein-protein complexes from a protein sequence data set. The method also includes performing featurization, using a featurization module, of the multi-dimensional structure of the plurality of protein-protein complexes by creating an adjacency matrix for successive spherical shells centered around each type of a plurality of amino acid sequences. The method further includes predicting, using a prediction module, a binding affinity from text sequence of the plurality of amino acid sequences using a pre-trained artificial intelligence model based on the adjacency matrix.

### BACKGROUND

### Technical Field

The present invention is generally related to the field of protein engineering. More particularly, the present invention is related to a method and system for predicting a binding affinity of protein structures based on deep learning

### Description of the Related Art

Generally, protein engineering involves the development of proteins that have certain biological activities. Antibodies are proteins which are usually generated by the body of organisms to defeat foreign agents, usually other protein structures called Antigens. With the enhancement of biomolecule development in the laboratory setting, techniques have been developed to generate artificial antibodies that can deal with specific antigens and also measure their activity towards those antigens. Notably, in-vitro antibody development is usually a very time-consuming process, as the possible combinations of amino acids to generate proteins are usually in the scale of thousands of trillions and scientists often apply heuristics and homological modeling to come up with proteins that are similar to natural proteins. With the development of deep learning techniques and in-silico docking techniques, attempts have been made to do the complete protein generation process in-silico, most often using natural language generation methods and text classification methods on protein and ligand sequences.

Typically, developing novel antibodies for antigen-binding tasks usually involves generating a large library of candidate antibody sequences and then filtering those generated sequences according to some rules and scores. Also, binding affinity is the most important score for such purposes, since In silico binding affinity prediction can render the antibody development process cheaper, faster and better, when compared to the wet lab methods. Although some works have emerged in recent years which claim to predict binding affinity, the existing techniques generalize poorly to proteins beyond their training dataset, mostly as they fail to use the complete information contained in the three-dimensional (3D) structure of the antibody-antigen complex. Also, existing techniques compute the binding affinity between proteins and ligands, that have a completely different structure and representation than proteins and also employ sequential computation which is time consuming and less efficient.

Hence there is need for a method and a system for effectively using the 3D structural information of the antibody-antigen complexes for binding affinity prediction or protein sequences.

The above-mentioned shortcomings, disadvantages and problems are addressed herein, and will be understood by reading and studying the following specification.

### SUMMARY

This summary is provided to introduce a selection of concepts in a simplified form that are further disclosed in the detailed description. This summary is not intended to determine the scope of the claimed subject matter.

The embodiments herein address the above-recited needs for a system and a method for predicting a binding affinity of protein structures based on deep learning based on multi-dimensional structural information of the antibody-antigen complexes for binding affinity prediction or protein sequences, that can be used for various applications such as, drug development and antibody affinity maturation and the like.

According to an aspect, a processor implemented method of predicting a binding affinity of protein structures based on deep learning is provided. The method includes capturing, using a capture module, a multi-dimensional structure of a plurality of protein-protein complexes from a protein sequence data set. The method also includes performing a featurization using a featurization module, of the multi-dimensional structure of the protein-protein complexes by creating an adjacency matrix for successive spherical shells centered around each type of amino acid sequences. The method also includes predicting, using a prediction module, a binding affinity from text sequence of the plurality of amino acid sequences using a pre-trained artificial intelligence model based on the adjacency matrix.

In an embodiment, the multi-dimensional structure of protein-protein complexes comprises three-dimensional coordinates of the atoms in the molecule along with corresponding chain name.

In an embodiment, the artificial intelligence model comprises a convolutional neural network (CNN) model.

In an embodiment, performing the featurization includes calculating a shell feature of each amino-acid pair comprising distances between the plurality of amino acids in the molecules and creating a plurality of feature vectors based on the number of amino acid pairs that fit in the interatomic distances.

In an embodiment, calculating the shell feature includes calculating a Euclidean distance between each atomic pair by calculating at least one of a minimum distance and a maximum distance between the plurality of amino acid sequences in a shell of a predetermined radius and a predetermined delta value, determining the shell feature based on the Euclidean distance for a predetermined inner sphere radius and shell thickness, and assigning a value of 1 to the feature upon the Euclidean distance being between the predetermined inner sphere and sum of the predetermined inner sphere and a delta value and assigning a value 0 to the feature upon the Euclidean distance being beyond the predetermined inner sphere and sum of the predetermined inner sphere and the delta value.

In an embodiment, the predicting the binding affinity comprises generating one or more PKA values indicative of the binding affinity by the pre-trained machine learning model based on the adjacency matrix, wherein the PKA values comprises one of a numerical value or a floating-point value.

In an embodiment, the adjacency matrix comprises intra and inter molecular distance values.

In an embodiment, predicting the binding affinity includes determining parent structures of the plurality of amino acid sequences using an artificial intelligence based model, generating protein 3D structures form the plurality of amino acid sequences by performing a homology modeling of the features of the plurality of amino acid sequences based on the parent structures and using the convolution neural network model, subjecting the multi-dimensional PDB structures of antibodies and antigens to a docking process, generating a PDB complex, and predicting the binding affinity of the plurality of amino acid sequences.

In another aspect, a method for training an artificial intelligence model for predicting a binding affinity of protein structures is provided. The method includes extracting a plurality of feature vectors from a protein sequence data set. The method also includes generating a training set for the artificial intelligence model based on the plurality of feature vectors and importing the training set into the artificial intelligence model. The method also includes training and evaluating the artificial intelligence model using the training set for predicting the binding affinity of protein structures.

In yet another aspect, a system for predicting a binding affinity of protein structures based on deep learning is provided. The system includes a non-transitory memory configured to store a protein sequence data set and one or more executable modules and a processor configured to execute the one or more executable modules for predicting a binding affinity of a plurality of protein structures. The one or more executable modules includes data capture module configured to capture a multi-dimensional structure of a plurality of protein-protein complexes from a protein sequence data set, a featurization module configured to perform the featurization of the multi-dimensional structure of the protein-protein complexes by creating an adjacency matrix for successive spherical shells centered around each type of amino acid sequences; and a prediction module configured to predict a binding affinity from text sequence of the amino acid sequences using a pre-trained artificial intelligence model based on the adjacency matrix.

In an embodiment, the multi-dimensional structure of protein-protein complexes comprises three-dimensional coordinates of the atoms in the molecule along with corresponding chain name.

In an embodiment, the artificial intelligence model comprises a convolutional neural network (CNN) model.

In an embodiment, the featurization module is further configured to calculate a shell feature of each amino-acid pair comprising distances between plurality of amino acids in the molecules and create a plurality of feature vectors based on the number of amino acid pairs that fit in the interatomic distances.

In an embodiment, the featurization module is further configured to calculate a Euclidean distance between each atomic pair by calculating at least one of a minimum distance and a maximum distance between the plurality of amino acid sequences in a shell of a predetermined radius and a predetermined delta value, determine the shell feature based on the Euclidean distance for a predetermined inner sphere radius and shell thickness, and assign a value of 1 to the feature upon the Euclidean distance being between the predetermined inner sphere and sum of the predetermined inner sphere and a delta value and assigning a value 0 to the feature upon the Euclidean distance being beyond the predetermined inner sphere and sum of the predetermined inner sphere and the delta value.

In an embodiment, the prediction module is further configured to generate one or more PKA values indicative of the binding affinity by the pre-trained machine learning model based on the adjacency matrix, wherein the PKA values comprises one of a numerical value or a floating-point value.

In an embodiment, the adjacency matrix comprises intra and inter molecular distance values.

In an embodiment, the prediction module is further configured to determine parent structures of the plurality of amino acid sequences using an artificial intelligence based model, generate protein 3D structures form the plurality of amino acid sequences by performing a homology modeling of the features of the plurality of amino acid sequences based on the parent structures and using the convolution neural network model, subject the multi-dimensional protein structures of antibodies and antigens to a docking process, generate a protein data bank complex, and predict the binding affinity of the plurality of amino acid sequences.

The method and system of the present technology makes the featurization roughly 21 times faster compared to other existing techniques as the featurization is implemented using the multiprocessing package in for example, python, which allows use of multiple processors in the same machine. Additionally, the method and system of the present technology employs inter and intra molecular shell based featurization that does not require knowledge of the corresponding chains each time manually (for each type of dataset) and performs better (generalizes better to test set) in practice when compared to intra-only feature matrix which is very sparse and contains very less info about the protein complex, especially with respect to the amount of information required to distinguish between small mutations of same parent molecule. Moreover, the method and system of the present technology employs homology modeling as a computational structure prediction method to determine protein 3D structure from its amino acid sequence. The present technology determines parent structures of the amino acid sequences using artificial intelligence (AI) based models such as Alphafold, Schrodinger, and the like. The use of AI based models to produce the parent structure and then using that structure to do homology modeling saves huge amount of time and computational resources.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

These and other objects and advantages will become more apparent when reference is made to the following description and accompanying drawings.

### BRIEF DESCRIPTION OF THE DRAWINGS

The other objects, features and advantages will occur to those skilled in the art from the following description of the preferred embodiment and the accompanying drawings in which:

FIG. 1 depicts an architecture of an implementation of system for predicting binding affinity of protein structures based on deep learning, according to one or more embodiments;

FIG. 2 depicts a pipeline for a process of predicting a binding affinity of protein structures based on deep learning, in accordance with an embodiment;

FIG. 3 depicts a structure of a protein data bank (PDB) file, in accordance with an exemplary scenario;

FIG. 4 depicts an example PDB file used for featurization by calculating shell feature for single amino acid pairs, in accordance with an exemplary scenario;

FIG. 5 illustrates parallelization of featurization process, in accordance with an exemplary scenario;

FIG. 6 depicts an example use of amino acids at both levels of nested loop, in accordance with an exemplary scenario;

FIG. 7 depicts shell based featurization, in accordance with an exemplary scenario;

FIG. 8 illustrates a flow diagram depicting a method of predicting a binding affinity of protein structures based on deep learning;

FIG. 9 illustrates a flow diagram depicting a method of training an artificial intelligence model for predicting a binding affinity of protein structure, in accordance with an embodiment; and

FIG. 10 depicts a representative hardware environment for practicing the embodiments herein.

Although the specific features of the embodiments herein are shown in some drawings and not in others. This is done for convenience only as each feature may be combined with any or all of the other features in accordance with the embodiments herein.

### DETAILED DESCRIPTION OF THE DRAWINGS

The detailed description of various exemplary embodiments of the disclosure is described herein with reference to the accompanying drawings. It should be noted that the embodiments are described herein in such details as to clearly communicate the disclosure. However, the amount of details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood, however, that it is not intended to limit the disclosure to the forms disclosed, but on the contrary, the disclosure is to cover all modifications, equivalents, and alternatives falling within the scope of the disclosure.

The detailed description of various exemplary embodiments of the disclosure is described herein with reference to the accompanying drawings. It should be noted that the embodiments are described herein in such details as to clearly communicate the disclosure. However, the details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood however, it is not intended to limit the disclosure to the forms disclosed, but on the contrary, the disclosure is to cover all modifications, equivalents, and alternatives falling within the scope of the disclosure.

The various embodiments of the present invention provide a method and a system for predicting binding affinity of protein structures based on deep learning. The method and system of the present technology is applicable in the field of drug development and discovery. In an embodiment, the method and system of the present technology enables computation of binding affinity between antibody and antigen molecules based on deep learning. Antibodies are proteins that protect you when an unwanted substance enters your body. Produced by the immune system, antibodies bind to these unwanted substances in order to eliminate them from your system. An antigen is a marker that tells your immune system whether something in your body is harmful or not. Antigens are found on viruses, bacteria, tumors and normal cells of your body. The present technology generates artificial intelligence models based on deep learning and shell based featurization that can classify protein sequences of antigens and antibodies in the scale of more than 1 million sequences for computing the binding affinity of the protein sequences, the information associated with which can be later used for drug development and discovery applications.

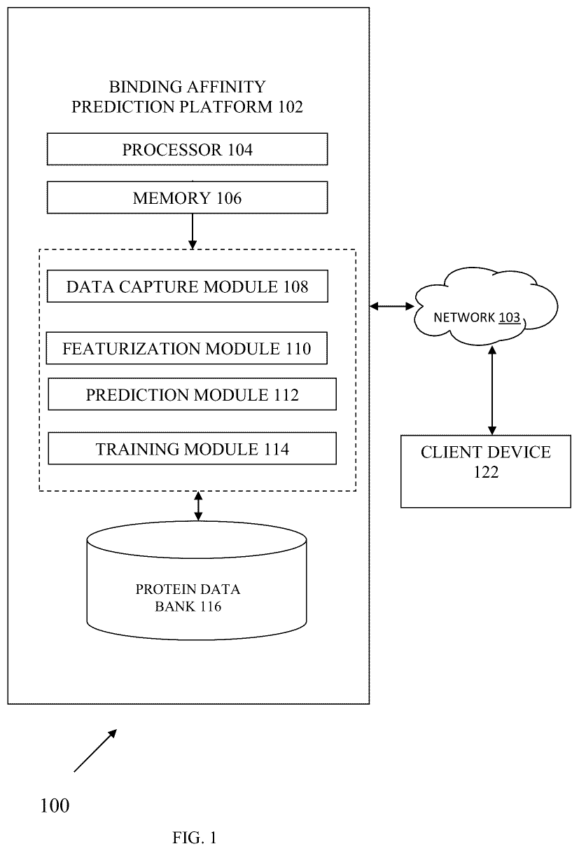

Referring to FIG. 1. FIG. 1 depicts an architecture of an implementation of system 100 for predicting binding affinity of protein structures based on deep learning, according to one or more embodiments. The system 100 may be a part of a server and may include binding affinity prediction platform 102 and a network 103 for enabling communication between the system components for information exchange. The network 103 may be for example, a private network and a public network, a wired network or a wireless network. The wired network may include, for example Digital Subscriber Line (DSL), coaxial cable lines, or optical fiber lines. The wireless network may include for example Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, a satellite band and other similar wireless networks. The wireless networks may also include any cellular network standards to communicate among mobile devices.

According to some embodiments, the binding affinity prediction platform 102 may be implemented in a variety of computing systems, such as a mainframe computer, a server, a network server, a laptop computer, a desktop computer, a notebook, a workstation, and the like. In an implementation, the binding affinity prediction platform 102 may be implemented in a server or in a computing device. In some embodiments, the binding affinity prediction platform 102 may be implemented as a part of a cluster of servers. In some embodiments, the binding affinity prediction platform 102 may be performed by the plurality of servers. These tasks may be allocated among the cluster of servers by an application, a service, a daemon, a routine, or other executable logic for task allocation.

In one or more embodiments, the binding affinity prediction platform 102 is configured to predict a binding affinity of protein-protein sequences based on a shell based featurization employing a parallel processing. As used herein the term “binding affinity” refers to a strength of the binding interaction between a single biomolecule (e.g., protein) to its binding partner. Typically, the cellular functions of proteins are maintained by forming diverse complexes and the stability of the protein complexes is quantified by the measurement of binding affinity, and mutations that alter the binding affinity can cause various diseases such as cancer and diabetes. As a result, accurate estimation of the binding stability and the effects of mutations on changes of binding affinity is a crucial step to understanding the biological functions of proteins and their dysfunctional consequences. Also, it has been hypothesized that the stability of a protein complex is dependent not only on the residues at its binding interface by pairwise interactions but also on all other remaining residues that do not appear at the binding interface. Most of the biological processes in cells are maintained by interactions between different proteins. Whether two specific proteins interact and how stable the interaction is are largely determined by the three-dimensional (3D) structures of these molecules, especially at the interface of the complex. The stability of a complex that is formed between two proteins can be quantified by their binding affinity. Therefore, accurate estimation of the binding affinity and the effects of mutations on changes of binding affinity is crucial to understanding the biological functions of proteins and their dysfunctional consequences. Relative to currently known traditional techniques, predicting binding affinity by computational methods is not only less time-consuming and labor-intensive but can also unravel the molecular mechanism of protein-protein interactions with details that are inaccessible through experimental measurements. The binding affinity prediction platform 102 of the present system 100 trains and tests machine learning models by construing a large set of molecular descriptors to calculate the binding affinity of protein-protein sequences.

According to some embodiments, binding affinity platform 102 may include processor 104 and memory 106. In an embodiment, the memory 106 may include a non-transitory memory configured to store a protein sequence data set and one or more executable modules. The processor 104 may be configured to execute the one or more executable modules for predicting a binding affinity of protein structures. In an embodiment, the one or more executable modules may include a data capture module 108, featurization module 110, prediction module 112, and training module 114. Further, binding affinity platform 102 may include a protein data bank (PDB) 116 storing data associated with all protein complexes, such as three-dimensional (3D) structure of the protein complexes, PDB index of the protein complexes, residue range, chain IDs, and the like.

According to some embodiments, the data capture module 108 is configured to capture a multi-dimensional (e.g., 3D) structure of a plurality of protein-protein complexes from a protein sequence data set. The protein sequence data set may be obtained from the PDB 116. In an embodiment, the multi-dimensional structure of protein-protein complexes includes three-dimensional (3D) coordinates of the atoms in the molecule along with corresponding chain names.

According to some embodiments, the featurization module 110 is configured to perform featurization of the multi-dimensional structure of the protein-protein complexes by creating an adjacency matrix for successive spherical shells centered around each type of amino acid sequences. As used herein the term “adjacency matrix” refers to a matrix used to represent finite graphs. The values in the matrix show whether pairs of nodes are adjacent to each other in the graph structure. If the graph is undirected, then the adjacency matrix will be a symmetric one. The adjacency matrix of proteins may include a matrix of shortest paths for protein graphs. The amino acid adjacency matrix includes a matrix representation of protein sequences leading to mathematical characterizations. The protein sequence, in this case, is directly translated into the matrix form without the intermediate graphical representation. The adjacency matrix comprises intra and inter molecular distance values, where the distance between atoms within same layer is also considered for computation. As used herein the term “featurization” refers to extraction and contextualization of the underlying structural features of protein sequences. The featurization may include, for example, contact boundaries, geometric transformations, graph networks of connectivity, and the like.

The 3D structure of protein-protein complexes is stored in text files called PDB files. It stores the x, y, z coordinates of atoms in the molecule along with the chain name, and other information. This information cannot be directly fed into neural network models and hence need to be converted into a usable format using different types of featurization techniques. The featurization module 110 creates an adjacency matrix for successive spherical shells centered around each type of amino acid. The featurization module 110 is further configured to calculate a shell feature of each amino-acid pair comprising distances between amino acids in the molecules and create a plurality of feature vectors based on the number of amino acid pairs that fit in the interatomic distances. The featurization module 110 is further configured to calculate a Euclidean distance between each atomic pair by calculating at least one of a minimum distance and a maximum distance between amino acid sequences in a shell of a predetermined radius and a predetermined delta value. The features are created based on how many amino acid pairs fit in the interatomic distances.

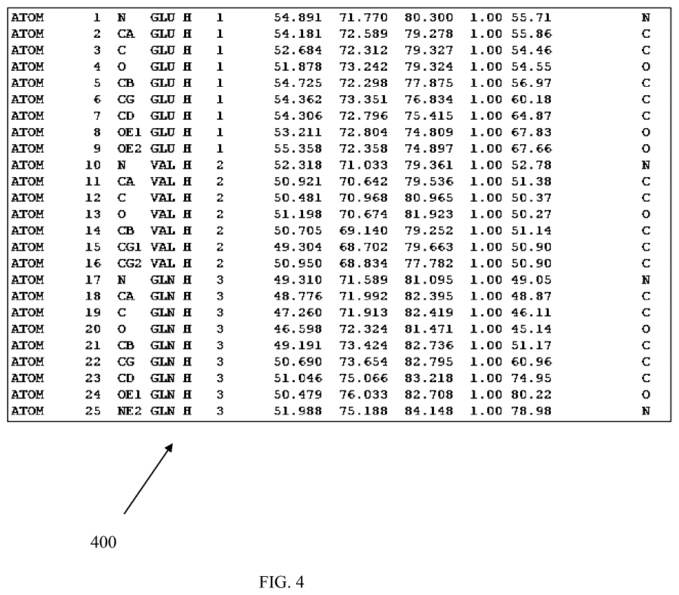

The featurization module 110 determines the shell feature based on the Euclidean distance for a predetermined inner sphere radius and shell thickness. The featurization module 110 assigns a value of 1 to the feature upon the Euclidean distance being between the predetermined inner sphere and sum of the predetermined inner sphere and a delta value and assigns a value 0 to the feature upon the Euclidean distance being beyond the predetermined inner sphere and sum of the predetermined inner sphere and the delta value. In an embodiment, the featurization is implemented using multi-processing package in python. Consider for example GLU and VAL for atoms 9 and 10 as shown in FIG. 4, the system 100 calculates a Euclidean distance between Atom 9 and Atom 10 given by sqrt((55.358−52.318)**2+(72.358−71.033)**2+(74.897−79.361)**2) which is equal to 5.56. Assuming inner sphere radius d of 4, and a shell thickness 8 of 0.5, then the above pair falls in the range of (4+0.5*3) and (4+0.5*4). Therefore, this adds 1 to the count in the feature at the fourth shell, feature[GLU_VAL_4]+=1.

During featurization, distance of each amino acid from all other amino acids is calculated. In serial processing, the distance between amino acid from all others, then the next amino acid with all other and so on is computed. In the present technology, the system 100 employs multiprocessing, to parallelize the above process. Accordingly, there will be a process computing distance of an amino acid with all other amino acids and there will be another parallel process executing simultaneously which will calculate distance of next amino acid with all other amino acids and so on. The parallelization facilitates decrease in time complexity by many folds.

According to some embodiments, the prediction module 112 is configured to predict a binding affinity from text sequence of the amino acid sequences using a pre-trained artificial intelligence model based on the adjacency matrix. In an embodiment, the artificial intelligence model includes a convolutional neural network (CNN) model. According to some embodiments, the prediction module 112 is further configured to determine a plurality of parent structures of the plurality of amino acid sequences using the artificial intelligence-based model (such as CNN). The prediction module 112 generates a plurality of multi-dimensional protein structures from the plurality of amino acid sequences by performing a homology modeling of the features of the amino acid sequences based on the parent structures and using the convolution neural network model. Homology modeling is one of the computational structure prediction methods that is used to determine protein 3D structure from its amino acid sequence. By employing homology modeling the prediction module 112 generates PDB file from protein sequences. The prediction module 112 subjects the multi-dimensional protein structures of antibodies and antigens to a docking process. Based on the docking process, the prediction module 112 generates a protein data bank complex and predicts the binding affinity of the plurality of amino acid sequences.

According to some embodiments, the training module 114 is configured to train an artificial intelligence model for predicting a binding affinity of protein structures. The training module 114 may be configured to extract a plurality of feature vectors from a protein sequence data set and generate a training set for the artificial intelligence model based on the plurality of feature vectors. The training module 114 may be configured to import the training set into the artificial intelligence model. The training module 114 may be configured to train and evaluate the artificial intelligence model using the training set for predicting the binding affinity of protein structures.

In an embodiment, the training is performed using CNN models. The CNN model is mainly used to deal with image features to build a deep Learning model. In an embodiment, three 2D-convolutional layers of sizes 64, 128 and 256 accompanied by a Relu (recurrent linear unit) activation function after each layer is used for training. Subsequently, three fully connected layers of size 200, 100 and 1, accompanied by Relu activation function, batchnorm and dropout at each layer except the last one is used along with linear activation for the last layer.

The system 100 may be accessible to a client device 122 via the network 103. Examples of the client device 122 includes, but is not limited to user devices (such as cellular phones, personal digital assistants (PDAs), handheld devices, laptop computers, personal computers, an Internet-of-Things (IOT) device, a smart phone, a machine type communication (MTC) device, a computing device, a drone, or any other portable or non-portable electronic device.

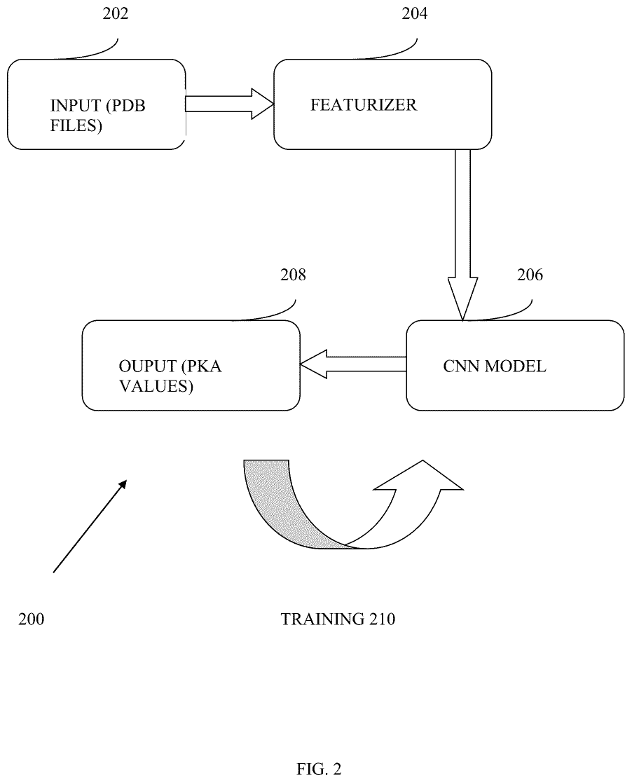

FIG. 2 depicts a pipeline 200 for a process of predicting a binding affinity of protein structures based on deep learning, in accordance with an embodiment. At stage 202 PDB files are received as input. The PDB files include a multi-dimensional structure of a plurality of protein-protein complexes from a protein sequence data set. At stage 204 the multi-dimensional structure of the protein-protein complexes is subjected to a featurization via a shell based featurization by creating an adjacency matrix for successive spherical shells centered around each type of amino acid sequence. At stage 206, a binding affinity is predicted from text sequence of the amino acid sequences using a pre-trained convolution neural network (CNN) model based on the adjacency matrix. At stage 208, the CNN model generates an output including PKA values indicative of binding affinity and can include either a numerical value or floating-point value. The CNN model is mainly used to deal with image features to build a deep learning model. In an embodiment, the CNN architecture uses three 2D-convolutional layers of sizes 64, 128 and 256 accompanied by Relu activation function after each layer. In an embodiment, three fully connected layers of size 200, 100 and 1, accompanied by Relu activation function, batchnorm and dropout at each layer except the last one is used. A linear activation is used for the last layer. In some embodiments, a modified CNN architecture may be used. The CNN model is trained 210 using the PKA values obtained by subjecting a training data set to the binding affinity prediction in the CNN model. The PKA values of amino acid side chains play an important role in defining the pH-dependent characteristics of a protein. The quantitative behavior of acids and bases in solution can be understood only if their PKA values are known. In particular, the pH of a solution can be predicted when the analytical concentration and PKA values of all acids and bases are known; conversely, it is possible to calculate the equilibrium concentration of the acids and bases in solution when the pH is known. These calculations find application in many different areas of chemistry, biology, medicine, and geology. For example, many compounds used for medication are weak acids or bases, and a knowledge of the PKA values. The PKA value is a number that describes the acidity of a particular molecule and measures the strength of an acid by how tightly a proton is held by a Bronsted acid. The lower the value of PKA, the stronger the acid and the greater its ability to donate its protons. describe the acidity of a particular molecule. KA denotes the acid dissociation constant and measures how completely an acid dissociates in an aqueous solution. The larger the value of KA, the stronger the acid as acid largely dissociates into its ions and has lower PKA value. The relationship between PKA and KA is described by the following equation (1). The PKA value is the negative base-10 logarithm of the acid dissociation constant (Ka) of a solution.

PKA=−log[Ka] (1)

The acid dissociation constants, or PKA values, are essential for understanding many fundamental reactions in chemistry. These values reveal the deprotonation state of a molecule in a particular solvent. The system 100 of the present technology uses a regression model which provides floating numbers in terms of PKA. The PKA values are used to find a threshold, above which the antibodies will have good binding affinity.

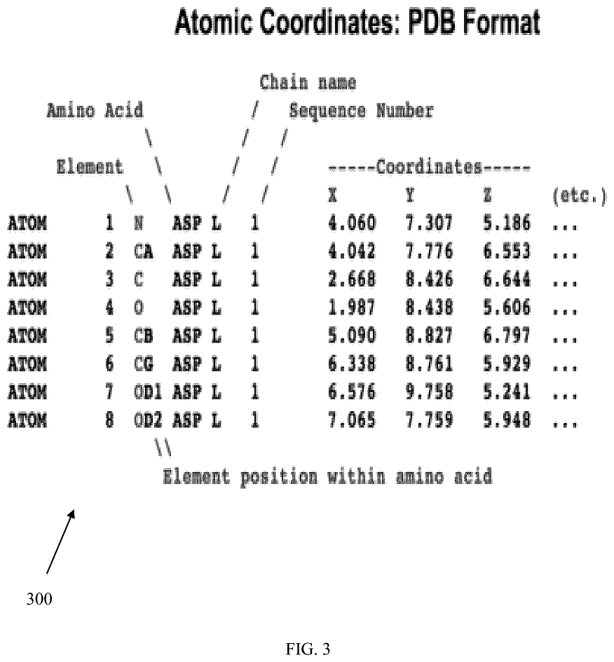

FIG. 3 depicts a structure 300 of a PDB file, in accordance with an exemplary scenario. As depicted in FIG. 3, the PDB file includes amino acids corresponding to each atom with a chain name, sequence number and x, y, and z coordinates corresponding to each atom and element position within each amino acid.

FIG. 4 depicts an example PDB file 400 used for featurization by calculating shell feature for single amino acid pairs, in accordance with an exemplary scenario. Consider for example GLU and VAL for atoms 9 and 10, the system 100 calculates a Euclidean distance between Atom 9 and Atom 10 given by sqrt((55.358−52.318)**2+(72.358−71.033)**2+(74.897−79.361)**2) which is equal to 5.56. Assuming inner sphere radius d of 4, and a shell thickness 8 of 0.5, then the above pair falls in the range of (4+0.5*3) and (4+0.5*4). Therefore, this adds 1 to the count in the feature at the fourth shell, feature[GLU_VAL_4]+=1.

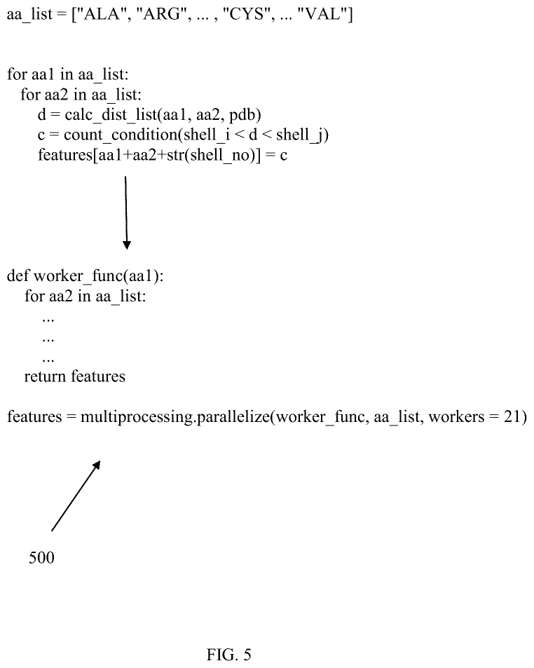

FIG. 5 illustrates parallelization 500 of featurization process, in accordance with an exemplary scenario. The parallelization renders the step of featurization to be around 21 times faster than conventional techniques. The parallelization is implemented using the multiprocessing package in, for example, python. The parallelization allows making use of multiple processors in the same machine. The parallelization is possible because the code contents of the nested loop given, do not have a sequential dependency.

The present system 100 uses inter and intra molecular shell based featurization instead of inter molecular shell alone. The intra-only feature matrix used in other existing techniques is very sparse and contains very less information about the protein complex, especially the amount of information required to distinguish between small mutations of the same parent molecule. The inter and intra molecular distances between atoms is used for shell based featurization in the present system 100 and various parts/shells that fall into same layer are considered for computation that enables extraction of more information and facilitates generalization for training and testing.

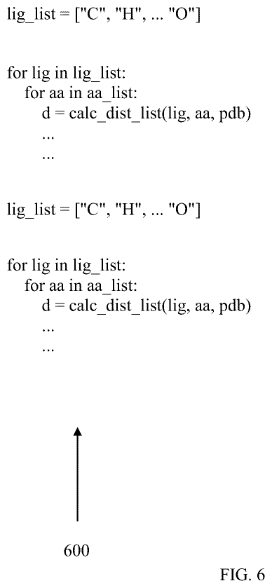

FIG. 6 depicts an example use of amino acids at both levels of nested loop 600, in accordance with an exemplary scenario. Typically, separate consideration of protein1 and protein2 requires knowledge of the corresponding chains each time manually (for each type of dataset), due to the information not being available in PDB files. Even if the information about chains is taken into consideration manually, the combined effect of intra and inter molecular interaction performs better (generalizes better to test set) in practice as intra-only feature matrix is very sparse and contains very less info about the protein complex, especially the amount of information required to distinguish between small mutations of same parent molecule.

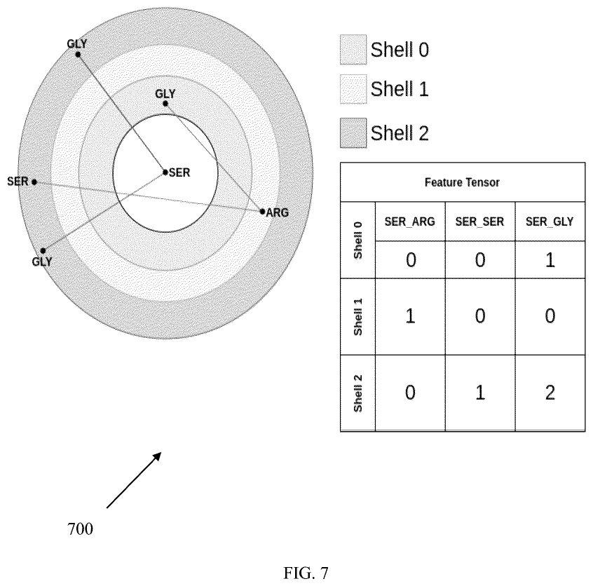

FIG. 7 depicts shell based featurization, in accordance with an exemplary scenario. As shown in FIG. 7, imaginary shells 700 of radius of 1 nano meter (nm) and thickness and delta 1 nm are considered in an exemplary scenario. The featurization module 110 calculates the minimum and maximum distance between amino acids. If the distances are between radius and radius+delta, the featurization module 110 assigns a value 1 to that feature, else it assigns a value 0. The featurization module 110 creates further spherical shells of radius r+delta and thickness delta and creates features. The advantage of the shell based featurization is that as many sizes of feature vectors can be created based on the needs of an application. Consider for example, 64 shells constituting 64 rows in the feature tensor with 21 unique amino acids. Which implies 21*21=441 unique pairs of amino acids constituting 441 columns in the feature tensor. A feature tensor of size 64*441 is obtained as an output of featurization and is passed into the CNN model for the prediction of binding affinity.

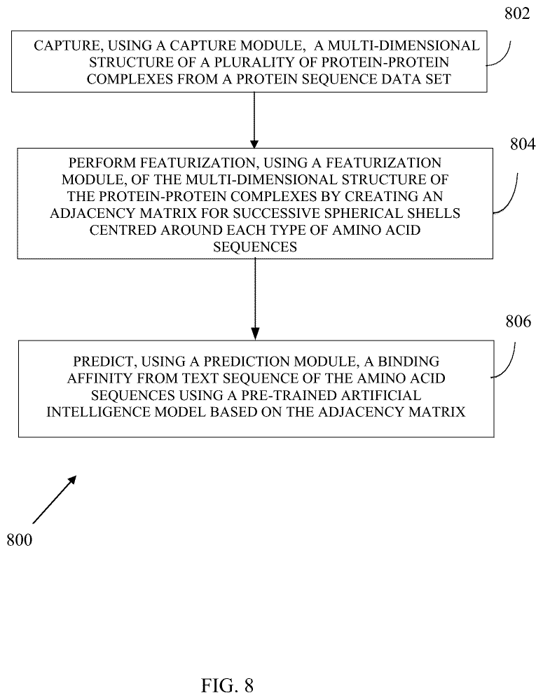

FIG. 8 illustrates a flow diagram 800 depicting a method of predicting a binding affinity of protein structures based on deep learning. At step 802, the method includes capturing, using a data capture module 108, a multi-dimensional structure of a plurality of protein-protein complexes from a protein sequence data set. At step 804, featurization of the multi-dimensional structure of the protein-protein complexes is performed using a featurization module 110, by creating an adjacency matrix for successive spherical shells centered around each type of amino acid sequences. In an embodiment, the adjacency matrix includes intra and inter molecular distance values. In an embodiment, the multi-dimensional structure of protein-protein complexes comprises three-dimensional coordinates of the atoms in the molecule along with corresponding chain name. In an embodiment, calculating the shell feature includes calculating a Euclidean distance between each atomic pair by calculating at least one of a minimum distance and a maximum distance between amino acid sequences in a shell of a predetermined radius and a predetermined delta value, determining the shell feature based on the Euclidean distance for a predetermined inner sphere radius and shell thickness and assigning a value of 1 to the feature upon the Euclidean distance being between the predetermined inner sphere and sum of the predetermined inner sphere and a delta value and assigning a value 0 to the feature upon the Euclidean distance being beyond the predetermined inner sphere and sum of the predetermined inner sphere and the delta value. In an embodiment, the featurization is implemented using multi-processing package in python.

At step 806, a binding affinity is predicted using a prediction module 112, from text sequence of the amino acid sequences using a pre-trained artificial intelligence model based on the adjacency matrix. In an embodiment, the artificial intelligence model comprises a convolutional neural network (CNN) model. In an embodiment, predicting the binding affinity includes determining parent structures of the amino acid sequences using an artificial intelligence based model, generating multi-dimensional (3D) protein structures from the plurality of amino acid sequences by performing a homology modeling of the features of the amino acid sequences based on the parent structures and using the convolution neural network model, subjecting the multi-dimensional PDB structures of antibodies and antigens to a docking process, generating a PDB complex, and predicting the binding affinity of the amino acid sequences. In an embodiment, the predicting the binding affinity comprises generating one or more PKA values indicative of the binding affinity by the pre-trained machine learning model based on the adjacency matrix, wherein the PKA values comprises one of a numerical value or a floating-point value.

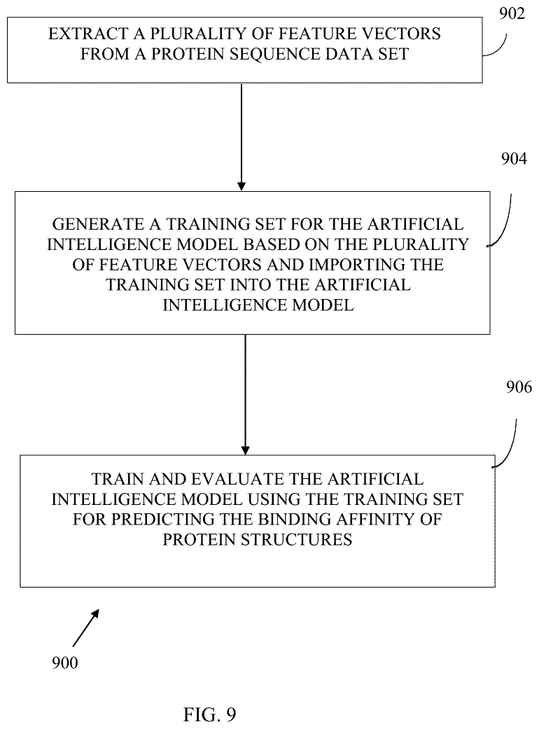

FIG. 9 illustrates a flow diagram 900 depicting a method of training an artificial intelligence model for predicting a binding affinity of protein structure, in accordance with an embodiment. At step 902, a plurality of feature vectors is extracted from a protein sequence data set. At step 904, a training set is generated for the artificial intelligence model based on the plurality of feature vectors and importing the training set into the artificial intelligence model. At step 906, the artificial intelligence model is trained and evaluated using the training set for predicting the binding affinity of protein structures.

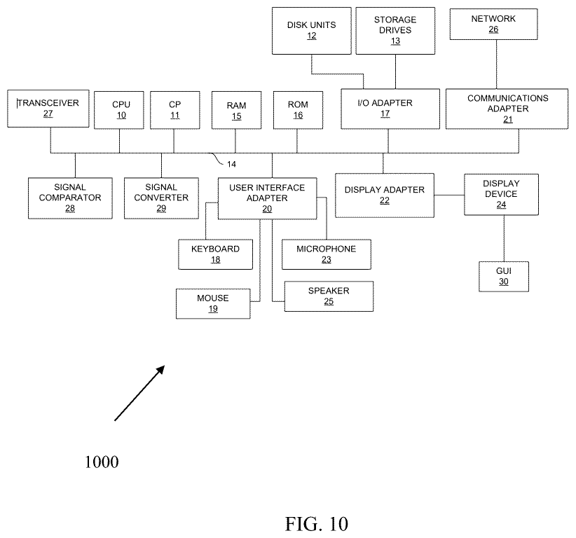

A representative hardware environment 1000 for practicing the embodiments herein is depicted in FIG. 10 with reference to FIGS. 1 through 9. This schematic drawing illustrates a hardware configuration of system 100 of FIG. 1, in accordance with the embodiments herein. The hardware configuration includes at least one processing device 10 and a cryptographic processor 11. The computer system 104 may include one or more of a personal computer, a laptop, a tablet device, a smartphone, a mobile communication device, a personal digital assistant, or any other such computing device, in one example embodiment. The computer system 104 includes one or more processor (e.g., the processor 108) or central processing unit (CPU) 10. The CPUs 10 are interconnected via system bus 12 to various devices such as a memory 14, read-only memory (ROM) 16, and an input/output (I/O) adapter 18. Although CPUs 10 are depicted, it is to be understood that the computer system 104 may be implemented with only one CPU.

The method and system of the present technology makes the featurization roughly 21 times faster compared to other existing techniques as the featurization is implemented using the multiprocessing package in for example, python, which allows use of multiple processors in the same machine. Additionally, the method and system of the present technology employs inter and intra molecular shell based featurization that does not require knowledge of the corresponding chains each time manually (for each type of dataset) and performs better (generalizes better to test set) in practice when compared to intra-only feature matrix which is very sparse and contains very less info about the protein complex, especially with respect to the amount of information required to distinguish between small mutations of same parent molecule. Moreover, the method and system of the present technology employs homology modeling as a computational structure prediction method to determine protein 3D structure from its amino acid sequence. The present technology determines parent structures of the amino acid sequences using an artificial intelligence-based models such as Alphafold, Schrodinger, and the like. The use of AI based models to produce the parent structure and then using that structure to do homology modeling saves huge amount of time and compute resources. Various embodiments of the present technology may be used for bio engineering fields where protein-protein binding affinity is required.

The embodiments herein (more particularly the executable modules including for example, the data capture module 108, the featurization module 110, the prediction module 112, and the training module 114) can take the form of, an entirely hardware embodiment, an entirely software embodiment or an embodiment including both hardware and software elements. The embodiments that are implemented in software include but are not limited to, firmware, resident software, microcode, and the like. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The system, method, computer program product, and propagated signal described in this application may, of course, be embodied in hardware; e.g., within or coupled to a Central Processing Unit (“CPU”), microprocessor, microcontroller, System on Chip (“SOC”), or any other programmable device. Additionally, the system, method, computer program product, and propagated signal may be embodied in software (e.g., computer readable code, program code, instructions and/or data disposed in any form, such as source, object or machine language) disposed, for example, in a computer usable (e.g., readable) medium configured to store the software. Such software enables the function, fabrication, modeling, simulation, description and/or testing of the apparatus and processes described herein.

Such software can be disposed in any known computer usable medium including semiconductor, magnetic disk, optical disc (e.g., CD-ROM, DVD-ROM, and the like) and as a computer data signal embodied in a computer usable (e.g., readable) transmission medium (e.g., carrier wave or any other medium including digital, optical, or analog-based medium). As such, the software can be transmitted over communication networks including the Internet and intranets. A system, method, computer program product, and propagated signal embodied in software may be included in a semiconductor intellectual property core (e.g., embodied in HDL) and transformed to hardware in the production of integrated circuits. Additionally, a system, method, computer program product, and propagated signal as described herein may be embodied as a combination of hardware and software

A “computer-readable medium” for purposes of embodiments of the present invention may be any medium that can contain, store, communicate, propagate, or transport the program for use by or in connection with the instruction execution system, apparatus, system or device. The computer readable medium can be, by way of example only but not by limitation, an electronic, magnetic, optical, electromagnetic, infrared, or semiconductor system, apparatus, system, device, propagation medium, or computer memory.

A “processor” or “process” includes any human, hardware and/or software system, mechanism or component that processes data, signals or other information. A processor can include a system with a general-purpose central processing unit, multiple processing units, dedicated circuitry for achieving functionality, or other systems. Processing need not be limited to a geographic location or have temporal limitations. For example, a processor can perform its functions in “real time,” “offline,” in a “batch mode,” etc. Portions of processing can be performed at different times and at different locations, by different (or the same) processing systems.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims. The scope of the embodiments will be ascertained by the claims to be submitted at the time of filing a complete specification.

What is claimed is:

1. A processor-implemented method of predicting a binding affinity of protein structures based on deep learning, the method comprising:
capturing, using a data capture module, a multi-dimensional structure of a plurality of protein-protein complexes from a protein sequence data set;
performing featurization, using a featurization module, of the multi-dimensional structure of the plurality of protein-protein complexes by creating an adjacency matrix for successive spherical shells centered around each type of a plurality of amino acid sequences; and
predicting, using a prediction module, a binding affinity from text sequence of the plurality of amino acid sequences using a pre-trained artificial intelligence model based on the adjacency matrix, by generating.

2. The processor-implemented method of claim 1, wherein the multi-dimensional structure of the plurality of protein-protein complexes comprises three-dimensional coordinates of the atoms in the molecule along with corresponding chain name.

3. The processor-implemented method of claim 1, wherein the artificial intelligence model comprises a convolutional neural network (CNN) model.

4. The processor-implemented method of claim 1, wherein performing featurization comprises:
calculating a shell feature of each amino-acid pair comprising distances between plurality of amino acid sequences in protein molecules; and
creating a plurality of feature vectors based on the number of amino acid pairs that fit in the interatomic distances.

5. The processor-implemented method of claim 3, wherein calculating the shell feature comprises:
calculating a Euclidean distance between each atomic pair by calculating at least one of a minimum distance and a maximum distance between the plurality of amino acid sequences in a shell of a predetermined radius and a predetermined delta value;
determining the shell feature based on the Euclidean distance for a predetermined inner sphere radius and shell thickness; and
assigning a value of 1 to the feature upon the Euclidean distance being between the predetermined inner sphere and sum of the predetermined inner sphere and a delta value and assigning a value 0 to the feature upon the Euclidean distance being beyond the predetermined inner sphere and sum of the predetermined inner sphere and the delta value.

6. The method of claim 1, wherein predicting the binding affinity comprises:
generating one or more PKA values indicative of the binding affinity by the pre-trained machine learning model based on the adjacency matrix, wherein the PKA values comprises one of a numerical value or a floating-point value.

7. The processor-implemented method of claim 1, wherein the adjacency matrix comprises intra and inter molecular distance values.

8. The processor-implemented method of claim 1, wherein predicting the binding affinity comprises:
determining a plurality of parent structures of the plurality of amino acid sequences using an artificial intelligence-based model;
generating a plurality of protein 3D structures form the plurality of amino acid sequences by performing a homology modelling of the features of the amino acid sequences based on the parent structures and using the convolution neural network model;
subjecting the multi-dimensional PDB structures of antibodies and antigens to a docking process;
generating a PDB complex; and
predicting the binding affinity of the plurality of amino acid sequences.

9. A processor-implemented method of training an artificial intelligence model for predicting a binding affinity of protein structures, the method comprising:
extracting a plurality of feature vectors from a protein sequence data set;
generating a training set for the artificial intelligence model based on the plurality of feature vectors and importing the training set into the artificial intelligence model;
training and evaluating the artificial intelligence model using the training set for predicting the binding affinity of protein structures.

10. A system for predicting a binding affinity of protein structures based on deep learning, the system comprising:
a non-transitory memory configured to store a protein sequence data set and one or more executable modules; and
a processor configured to execute the one or more executable modules for predicting a binding affinity of protein structures, wherein the one or more executable modules comprises:
a data capture module configured to capture a multi-dimensional structure of a plurality of protein-protein complexes from a protein sequence data set;
a featurization module configured to perform featurization of the multi-dimensional structure of the plurality of protein-protein complexes by creating an adjacency matrix for successive spherical shells centered around each type of amino acid sequences; and
a prediction module configured to predict a binding affinity from text sequence of the amino acid sequences using a pre-trained artificial intelligence model based on the adjacency matrix.

11. The system of claim 10, wherein the multi-dimensional structure of plurality of protein-protein complexes comprises three-dimensional coordinates of the atoms in the molecule along with corresponding chain name.

12. The system of claim 10, wherein the artificial intelligence model comprises a convolutional neural network (CNN) model.

13. The system of claim 10, wherein the featurization module is further configured to:
calculate a shell feature of each amino-acid pair comprising distances between a plurality of amino acids in the molecules; and
create a plurality of feature vectors based on the number of amino acid pairs that fit in the interatomic distances.

14. The system of claim 10, wherein the featurization module is further configured to:
calculate a Euclidean distance between each atomic pair by calculating at least one of a minimum distance and a maximum distance between a plurality of amino acid sequences in a shell of a predetermined radius and a predetermined delta value;
determine the shell feature based on the Euclidean distance for a predetermined inner sphere radius and shell thickness; and
assign a value of 1 to the feature upon the Euclidean distance being between the predetermined inner sphere and sum of the predetermined inner sphere and a delta value and assigning a value 0 to the feature upon the Euclidean distance being beyond the predetermined inner sphere and sum of the predetermined inner sphere and the delta value.

15. The system of claim 10, wherein the prediction module is further configured to:
generate one or more PKA values indicative of the binding affinity by the pre-trained machine learning model based on the adjacency matrix, wherein the PKA values comprises one of a numerical value or a floating-point value.

16. The system of claim 10, wherein the adjacency matrix comprises intra and inter molecular distance values.

17. The system of claim 10, wherein the prediction module is further configured to:
determine parent structures of the plurality of amino acid sequences using an artificial intelligence-based model;
generate a plurality of multi-dimensional protein structures from the plurality of amino acid sequences by performing a homology modeling of the features of the plurality of amino acid sequences based on the parent structures and using the convolution neural network model;
subject the multi-dimensional protein structures of antibodies and antigens to a docking process;
generate a protein data bank complex; and
predict the binding affinity of the plurality of amino acid sequences.
